(19) United States
(12) Patent Application Publication
(10) Pub. No.: US 2004/0191815 A1
(43) Pub. Date: Sep. 30, 2004

(54) Array having oligonucleotides on a metal substrate

(76) Inventors: Motoki Kyo, Tsuruga-shi (JP); Masayuki Yamamoto, Tsukuba-shi (JP); Hozumi Motohashi, Tsukuba-shi (JP); Kinuko Ohneda, Tsukuba-shi (JP); Yutaka Takarada, Tsuruga-shi (JP); Bunsei Kawakami, Tsuruga-shi (JP); Yoshihisa Kawamura, Tsuruga-shi (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(21) Appl. No.: 10/756,767

(22) Filed: Jan. 14, 2004

(30) Foreign Application Priority Data
Jan. 15, 2003 (JP) 2003-7469
Jan. 15, 2003 (JP) 2003-7482
Aug. 21, 2003 (JP) 2003-298027
Sep. 25, 2003 (JP) 2003-333072

(51) Int. Cl. C12Q 1/68; C07H 21/04; C12M 1/34

(52) U.S. Cl. 435/6; 435/287.2; 536/24.3

(57) ABSTRACT

The array of the present invention has a plurality of double-stranded oligonucleotides immobilized on a metal substrate. Each of the double-stranded oligonucleotides includes a first single-stranded oligonucleotide and a second single-stranded oligonucleotide. The first and second single-stranded oligonucleotides are entirely or partially bonded together in a complementary manner to form said double-stranded oligonucleotide. Among the first and second single-stranded oligonucleotides, only the first single-stranded oligonucleotide is bonded on said substrate. The present invention also relates to a method for analyzing the interaction between the immobilized biomolecules and another biomolecule or an aggregate thereof by use of the array.

CROSS-REFERENCE TO RELATED APPLICATION

The present application is based on and claims priority of Japanese patent applications No. 2003-7469 filed on Jan. 15, 2003, No. 2003-7482 filed on Jan. 15, 2003, No. 2003-298027 filed on Aug. 21, 2003 and No. 2003-333072 filed on Sep. 25, 2003, the entire contents of which are hereby incorporated by reference.

### FIELD OF THE INVENTION

The present invention relates to an array comprising double-stranded oligonucleotides immobilized thereto and a method for immobilizing biomolecules to a solid surface and measuring the interaction between the immobilized biomolecules and another biomolecule or an aggregate thereof. The present invention also relates to a method for analyzing the interaction between the immobilized biomolecules and another biomolecule or an aggregate thereof by use of the array.

### BACKGROUND ART

Heretofore, DNA array technologies have been used as a method for detecting genes. Generally, in the DNA array technologies, a single-stranded DNA is immobilized on a substrate, and it is detected whether a nucleic acid complementary to the DNA is hybridized thereto, by means of a fluorescent-labeling or chemiluminescence method.

In recent years, a great interest has been devoted to the observation of the interaction between different biomolecules, such as DNA-protein interaction, as well as the interaction between nucleic acids. One of the reasons is that the existence of a protein specific to a nucleic acid sequence has become widely known. In particular, an interaction of association/dissociation is regarded as important.

The association/dissociation interaction between DNA and protein has been commonly evaluated by a gel mobility shift assay in which the relative mobility of the DNA and protein in a gel are observed while maintaining the interaction therebetween. However, the gel mobility shift assay involves difficulties when handling a large number of samples due to its low throughput. Further, it is impossible to evaluate the association/dissociation rate because the measurement is performed in an equilibrium state.

In these circumstances, research has been made to develop a method for analyzing the interaction with proteins using a double-stranded DNA array prepared by utilizing the DNA array technologies. For example, a method has been known comprising immobilizing single-stranded DNAs on a solid substrate in an array arrangement, hybridizing to each of the DNAs a primer identical to the primer site of the DNA, and performing DNA polymerase synthesis on the surface of the substrate to form double-stranded DNAs (U.S. Pat. No. 6,326,489). While this method is effective if the DNA to be immobilized is a single long strand having a primer site identical to that of a primer to be hybridized thereto, it involves complexity in optimization of the conditions of the DNA polymerase reaction on the substrate and manipulation of the reaction. In U.S. Pat. No. 6,326,489, a protein is labeled with Green Fluorescent Protein (GFP) or the like to observe the interaction with the double-stranded DNA. However, the labeling cannot be considered as a method capable of universally measuring DNA-protein interactions, because the functions or characteristics of the protein are likely to be changed by the labeling.

There has also been reported a method comprising forming a double-stranded DNA through hybridization instead of performing the enzymatic synthesis on the substrate, immobilizing the DNA on the substrate, and observing the interaction between the immobilized DNA and an enzyme (Boon et al., Nature Biotechnology 20 (2002) 282-286). In this method, a thiol group having an affinity to a gold substrate is introduced into a DNA molecule, and then the DNA molecule is immobilized directly on the gold substrate. This method is intended to immobilize the double-stranded DNAs on the entire area of an electrode, and any concept using an array is not suggested therein. It is known that some of the DNA molecules immobilized using this method lay down on the gold substrate (Tonya et al., J. Am. Chem. Soc. 119 (1997) 8916-8920). In this case, the DNA molecules located excessively close to the substrate cannot maintain their sufficient mobility, which may cause difficulties in accurately measuring the interaction kinetics with proteins. Further, if the DNA molecules are immobilized on the gold substrate by itself through self-assembly, they cannot be densely packed on the surface of the gold substrate. Thus, it is required to use nonionic alkanethiol, for example, mercaptohexanol, to block the unnecessary contact between the DNA molecule and the gold substrate. A stronger self-assembling ability of mercaptohexanol causes an exchange reaction with the previously immobilized DNA molecules to seriously reduce the density of the DNA molecules finally left on the surface of the gold substrate. Consequently, the obtained DNA molecules can provide only an extremely weak signal from the interaction with proteins.

When the mixture of thiol-terminated DNA and mercaptohexanol is applied on the gold surface, the density of the DNA molecules is small and is hard to be controlled by the mixture ratio change.

A method for observing a DNA-protein interaction on an array has also been reported (Brockman et al., J. Am. Chem. Soc. 121 (1999) 8044-8051). This method comprises immobilizing two different single-stranded DNA groups, exposing DNAs complementary to one of the groups to the entire array to hybridize the complementary DNA only to the one group, and observing the interaction with a single-strand binding (SSB) protein through surface plasmon resonance (SPR) imaging. In this method, if the two DNA groups to be immobilized are analogous in sequence, even a mismatched DNA can be hybridized thereto. Thus, it is difficult to prepare an array of different double-stranded DNA groups analogous in sequence. There is also a limit to the range of exposing the complementary DNAs to the DNA array or DNA chips.

The biomolecule interaction has also been checked to figure out the functions of a biomolecule. Typically, the biomolecule interaction is checked by immobilizing a reference molecule on the surface of a solid substrate, and bringing a target substance into contact with the reference molecule to detect whether the target substance is absorbed into the reference molecule. During the measurement of the interaction, it is desired to inhibit non-specific absorption while ensuring the mobility of the immobilized molecule. For this purpose, it has been considered to essentially provide a spacer for maintaining a space between the molecule to be immobilized and the surface of the substrate. For example, there has been known a method for immobilizing a nucleic acid molecule on a solid substrate through covalent bonding, in which a spacer consisting of 16-base thymine or polyethylene glycol is linked to the nucleic acid molecule (WO 00/67028). A crosslinking agent used for covalent bonding in this method has no function as a spacer, and the resulting insufficient mobility of the immobilized molecule is a factor limiting substances which may be subjected to the interaction analysis.

A method using a polymer as the spacer has also been reported (Japanese Patent Laid-Open Publication No. 2000-146976). The polymer used in this method is a multifunctional polymer having a number of amino groups, and thus not effective to ensure a sufficient distance from the surface of the substrate. Further, if the functional groups are located at both terminals of the polymer, both terminals will cause reactions to decrease the efficiency of reaction with bio-substances.

U.S. Pat. No. 5,436,161 discloses a method comprising forming a hydrogel on the surface of a substrate, and introducing a functional group into the gel to ensure the mobility of a molecule bound to the gel so as to inhibit non-specific absorption. While this method is effective to inhibit non-specific absorption, it is unsuitable to accurately measure interaction kinetics in real time, due to the variations in penetration/diffusion rate of a target substance between the inside and outside regions of the gel.

There has also been reported a spacer comprising hydrophilic polymer molecules. The spacer is designed to have one terminal having a functional group for immobilizing thereto a biomolecule for use in interaction observation, and the other terminal having a functional group capable of bonding directly to the surface of a solid substrate (Sigal et al. Anal. Chem. 68 (1996) 490-497, Jung et al. Langmuir, 16, (2000) 9421-9432). This spacer molecule cannot be densely packed on the surface of the gold substrate by itself, and consequently the terminal of the spacer molecule has to be combined with an additional hydrophilic group at a high ratio to inhibit non-specific absorption. This causes problems of low density of the biomolecules to be immobilized to the surface of the solid substrate, and deterioration in measurement sensitivity.

WO 01/86301 also discloses a technique of applying polyethylene glycol to the surface of a biosensor.

Another conventional technology includes a biochip array having a background area with polyethylene glycol immobilized thereto (U.S. Pat. No. 6,127,129, Prime et al. J. Am. Chem. Soc. 115, (1993) 10714-10721), and a biosensor having a surface with hydrophilic polymer molecules immobilized thereto (Anal. Biochem. 198, 268, (1991), U.S. Pat. No. 6,127,129).

### SUMMARY OF THE INVENTION

In view of the above circumstances, it is therefore an object of the present invention to provide an array comprising immobilized double-stranded oligonucleotides, and a method for adequately measuring the interaction between biomolecules.

It is another object of the present invention to provide a method capable of accurately analyzing interaction kinetics while ensuring the mobility of molecules immobilized to an array and inhibiting non-specific absorption.

As used in the specification, the term “array” means a plurality of regions formed in an array arrangement to immobilize double-stranded oligonucleotides thereto, wherein each of the regions may be in the state after the double-stranded oligonucleotides have been already immobilized thereto or may be in the state before the double-stranded oligonucleotides are immobilized thereto.

### BRIEF DESCRIPTION OF THE DRAWINGS

FIG. 1 shows the base sequence of MARE 25.

FIG. 2 shows the base sequence of MARE 23.

FIG. 3 is a photograph showing an SPR image of a dsDNA array.

FIG. 4 is a graph showing the change of an SPR signal (association/dissociation curve of a MafG homodimer) in Inventive Example 1.

FIG. 5 is a photograph showing the difference between images before and after the association of a MafG homodimer.

FIG. 6 is a graph showing the change of an SPR signal (association/dissociation curve of a MafG homodimer) in Referential Example 1.

FIG. 7 is a diagram showing a photomask used in Inventive Example 2.

FIG. 8 is a graph showing the change of an SPR signal in Inventive Example 2.

FIG. 9 is a graph showing the change of an SPR signal when poly-L-lysine is supplied in Referential Example 2.

FIG. 10 is a graph showing the change of an SPR signal when poly-L-lysine is supplied in Referential Example 3.

FIG. 11 is a step diagram showing the steps for preparing an array in Inventive Example 2.

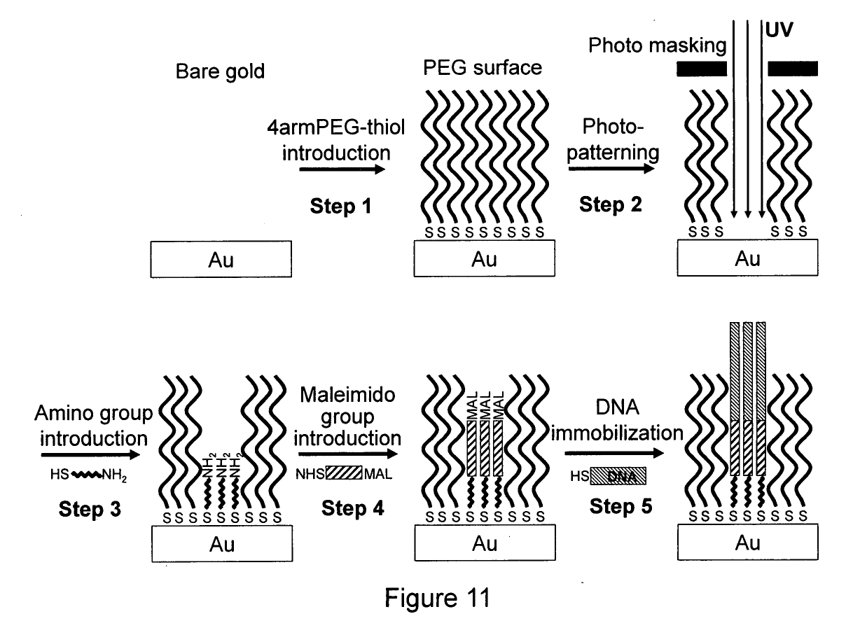

FIG. 12 is a diagram showing the scheme of the reaction of a crosslinking agent in Inventive Example 2.

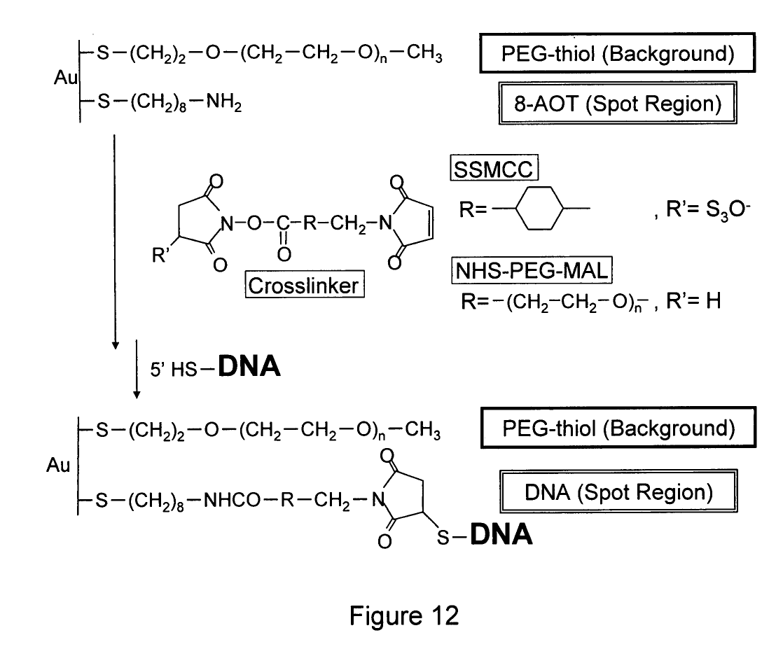

FIG. 13 is a graph showing the change of an SPR signal in Inventive Example 3.

FIG. 14 is a photograph showing the change in image in Inventive Example 3.

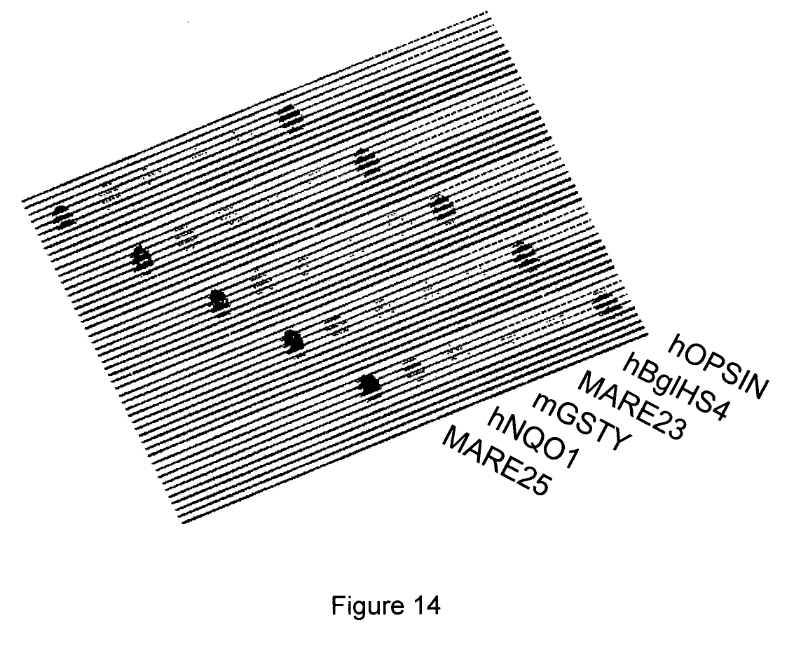

### DESCRIPTION OF PARTICULAR EMBODIMENTS

Particular embodiments of the present invention will now be described in detail.

As used in the specification, the term “double-stranded oligonucleotide” means a double-stranded DNA or double-helix molecule of DNA and RNA. In the present invention, a plurality of double-stranded oligonucleotides are immobilized on an array. The double-stranded oligonucleotide in the present invention is formed by entire or partial bonding between a first oligonucleotide and a second oligonucleotide in a complementary relation. The term “partial” means that a portion of either one of the first and second oligonucleotides is a single strand or a mismatched base pair.

In the array of the present invention, the double-stranded oligonucleotide comprises two single-stranded oligonucleotides, wherein only one of the single-stranded oligonucleotides (first oligonucleotide) is bonded onto a metal substrate, and the other oligonucleotide (second oligonucleotide) is immobilized onto the substrate through the first oligonucleotide in the form of a double-stranded oligonucleotide by forming a Watson-Crick pair complementarily with the first oligonucleotide.

The first and second oligonucleotides bonded together in a complementary relation are set to have a melting point (Tm) greater than a measurement temperature. While the melting point Tm is dependent on the percentages of G and C included in the oligonucleotides, it is preferably set so as to allow a complementary bonding of at least nine or more bases to be created at a commonly used measurement temperature (25 to 37° C.). A long base pair of fifty or more bases causes the risk of difficulty in synthesis, creation of a self-complementary base pair, occurrence of hybridization at a site different from a target site, or non-formation of an intended double-stranded oligonucleotide. Thus, the complementarily bonded site in the double-stranded oligonucleotide has preferably a length in the range of nine bases to fifty bases, more preferably eleven bases to thirty bases. Preferably, the complementarily bonded bases are continuous. As long as these bases are complementarily bonded together, the complementarily bonded site herein may partially include a mismatched base pair.

In the observation of the interaction with a biomolecule, it is required to allow the biomolecule to recognize the oligonucleotide. For this purpose, it is critical to bond the double-stranded oligonucleotide molecule on the substrate, not at a central position but at the terminal of the oligonucleotide, so as to prevent the double-stranded oligonucleotide molecule from laying down on the substrate. For example, a process of bonding the oligonucleotide at the terminal thereof may comprise introducing a functional group to the terminal of the first oligonucleotide, and bonding the introduced functional group directly or indirectly using a cross-linking agent with a functional group residing on the solid surface or a functional group introduced onto the solid surface. The double-stranded oligonucleotide immobilized at the terminal thereof in this way allows a biomolecule to adequately recognize the oligonucleotide so as to provide a correct evaluation value.

Process of Preparing Double-Stranded Oligonucleotide Array

Preferably, a double-stranded oligonucleotide array is prepared, without being limited thereto, through a process comprising hybridizing a plurality of first oligonucleotides and a plurality of second oligonucleotides to form a plurality of double-stranded oligonucleotides, an then immobilizing the respective terminals of the first oligonucleotides to a metal substrate in an array arrangement.

More specifically, the process preferably comprises the steps of (1) hybridizing a first single-stranded oligonucleotide and a second single-stranded oligonucleotide to form a double-stranded oligonucleotide having the first and second single-stranded oligonucleotides entirely or partially bonded together in a complementary manner, and (2) bonding a terminal of the first single-stranded oligonucleotide on a metal substrate to immobilize the double-stranded oligonucleotide formed in step (1) on the metal substrate.

If the first single-stranded oligonucleotide is first immobilized onto the metal substrate, and the second single-stranded oligonucleotide is then hybridized to the first single-stranded oligonucleotide, it will be required to locate or spot the first and second single-stranded oligonucleotides at the same position of the array, or to perform a spotting operation two times. This causes complexity in process. Thus, before any spotting operation, the hybridization between the first and second single-stranded oligonucleotides is preferably performed to form the double-stranded oligonucleotide.

The hybridization between the first and second single-stranded oligonucleotides is performed, for example, through a process comprising adding a 5′-thiol-terminated DNA and a complementary DNA to a solution, such as ×5SSC solution, having a high salt concentration at the mol ratio of the 5′-thiol-terminated DNA to the complementary DNA ranging from 1:1 to 1:10, incubating the mixture for 3 to 15 minutes while keeping the mixture boiling, rapidly cooling the mixture at 0° C. to incubate it for 5 to 60 minutes, and then heating the mixture up to 37° C. to incubate it for 1 to 24 hours.

The first oligonucleotide is bonded on the metal substrate, preferably, through a process comprising introducing a functional or bonding group to the terminal of the first oligonucleotide, and bonding the functional or bonding group introduced to the first oligonucleotide directly on the substrate or indirectly on the substrate through an intervening substance. The functional or bonding group includes, but is not limited to, amino group, thiol group, aldehyde group, maleimide group and biotin.

The metal substrate is made, for example, of gold, silver, copper, aluminum or chromium. In particular, a transparent substrate having a surface layer formed of a thin gold layer is preferably used as the metal substrate. If the thin gold layer is formed on the surface of a substrate, after a specific substance is densely packed on the surface to introduce functional groups onto the gold surface, the respective terminals of the first oligonucleotides can be bonded directly or indirectly with the functional groups of the specific substance to densely immobilize the oligonucleotides to the substrate.

The functional groups are introduced onto the gold surface, preferably, through a process of reacting bifunctional group-type alkane expressed by a general formula X′—R′—Y′ (wherein X′ is a functional group to be bonded with the gold surface, Y′ being a functional group to be bonded with the first oligonucleotide, and R′ being an organic group, wherein R′ may be bonded to the first oligonucleotide through a cross-linking agent, as described later) with the gold surface as a surface-treating or coupling agent to densely pack the bifunctional group-type alkane on the surface of the substrate. The functional group X′ may include thiol group, sulfide group and disulfide group. The functional group Y′ may include amino group, carboxyl group, aldehyde group, azido group, N-hydroxysuccinimide group, epoxy group, carbonyl imidazole group and isocyanate group. The organic group R′ may include alkylene group. Preferably, the carbon number of the alkylene group is in the range of 5 to 18, without being limited thereto. If the carbon number is less than 5, the hydrophobic bonding of alkane chain becomes strong to cause not only the difficulty in reaction/immobilization of a hydrophilic substance but also the risk of occurrence of non-specific absorption during measurement due to a strong hydrophobic property of the sensor surface.

Specifically, the compound expressed by the general formula X′—R′—Y′, may include 8-amino-1-octanethiol wherein Y′ is an amino group, and 7-carboxy-1-heptanethiol wherein Y′ is a carboxyl group.

Preferably, the compound of the general formula X′—R′—Y′ on the substrate is bonded with the first oligonucleotide through a cross-linking agent. This point will be described in detail later.

Process of Measuring Biomolecule Interaction

The double-stranded oligonucleotides of the present invention can be suitably used in the measurement of the interaction between the oligonucleotides and a biomolecule or aggregate thereof. The measurement of the interaction is preferably performed using a surface plasmon resonance (SPR) method allowing a label-free and real-time measurement. In the SPR method, an SPR imaging is particularly preferable.

Labeling causes changes in functions/activity of some biomolecules. In particular, proteins have a high risk of causing structural change or inactivation due to the labeling. The SPR method allows any interaction analysis to be performed without labeling biomolecules, to achieve an adequate measurement while maintaining the functions and activities of biomolecules. In addition, according to the SPR method, the measurement can be performed in real time even during a transient state as well as an equilibrium state to allow the association/dissociation rate to be analyzed. Thus, the obtained measurement results can provide valuable information for figuring out the functions of biomolecules.

In the SPR imaging, the whole area of the array is irradiated with polarized parallel rays, and the resulting reflected light is picked up by a CCD camera. Thus, a surface-plasmon-resonance color gradation at a certain position of the array can be figured out according to changes in the reflected light. Therefore, the SPR imaging allows the interaction between the immobilized double-stranded oligonucleotide having a plurality of sequences and a biomolecule to be measured or analyzed in real time without labeling, and can be suitably used in analyzing the biomolecule interaction using an array having a plurality of biomolecules immobilized thereon.

A biomolecule subject to interaction with the oligonucleotides includes, without being limited thereto, nucleic acid, protein, peptide and sugar chain. It may also include an aggregate of biomolecules such as a heterodimer. In this manner, the present invention can be suitably used in the measurement of proteins, particularly, transfer factors having a known function of interacting with a double-stranded DNA. The transfer factors include, without being limited thereto, a NFI family, Maf family and GATA family. It is known that the Maf family forms not only homodimers but also heterodimers. According to the present invention, the evaluation of the difference between the bonding behaviors of various combinations of heterodimers, and the analysis of the interaction between a modified double-stranded DNA and a transfer factor can be performed to obtain various useful data regarding biofunctions.

In the present invention, a process of measuring biomolecule interaction includes a step of measuring the interaction between a biomolecule (A) and a biomolecule (B) or an aggregate thereof, using a solid substrate, wherein the biomolecule (A) is immobilized on a solid surface of the substrate by use of a cross-linking agent consisting of a heterobifunctional hydrophilic polymer molecule expressed by a general formula X—R—Y (wherein: X is a functional group on the solid surface or a functional group to be bonded with a functional group introduced onto the solid surface; Y being a functional group to be bonded with the biomolecule (A); and R being a repeating unit of the polymer molecule, wherein X and Y are preferably two functional groups selected from the group consisting of amino group, carboxyl group, succinimide group, sulfonated succinimide group, maleimide group, thiol group, aldehyde group, vinyl group, isocyanate group, epoxy group, hydrazine group and azido group, wherein X≠Y).

The present invention will be described in more detail below.

Heterobifunctional Hydrophilic Polymer Molecule

In the present invention, a heterobifunctional hydrophilic polymer molecule is preferably used as the cross-linking agent in the process of immobilizing a biomolecule onto a solid surface.

According to this process, the biomolecule can be immobilized while inhibiting non-specific absorption and maintaining a certain space from the surface. Thus, the mobility of the biomolecule can be ensured to allow the analysis of biomolecule interaction kinetics to be accurately performed through a real-time and adequate observation.

The heterobifunctional hydrophilic polymer molecule used as the cross-linking agent is expressed by the general formula X—R—Y. In this formula, X is a functional group on the solid surface or a functional group to be bonded with a functional group introduced onto the solid surface. Y is a functional group to be bonded with the biomolecule (A). R is a repeating unit of the polymer molecule. The polymer molecule herein has a repeating unit of 3 or more.

The two different functional groups residing at the terminals of the polymer molecule serving as the cross-linking agent comprise one functional group X on a solid surface or a functional group to be bonded with a functional group introduced onto the solid surface, and a functional group Y to be bonded with the biomolecule (A). That is, these two functional groups X, Y are different from each other, or X≠Y.

Preferably, the functional group Y is not reactive to the solid surface. If the functional group Y reacts with the surface, a loop will be undesirably formed on the surface to reduce the number of functional groups for immobilizing the biomolecule (A), resulting in deteriorated reaction efficiency.

The functional groups X, Y include amino group, carboxyl group, succinimide group, sulfonated succinimide group, maleimide group, thiol group, aldehyde group, vinyl group, isocyanate group, epoxy group, hydrazine group and azido group. Two different functional groups may be selected from the above functional groups, and used in combination as functional groups X, Y.

The combination of an amino group and a carboxyl group each reacting with a different functional group, or the combination of a succinimide group and a maleimide group each reacting with a different functional group, is particularly preferable as the combination of X and Y.

When the biomolecule (A) is a DNA, the functional group Y to be introduced in the terminal of the polymer molecule is preferably an amino group, thiol group or biotin.

The molecular weight of the heterobifunctional hydrophilic polymer molecule is in the range of 200 to 20000, preferably 1000 to 10000, more preferably 1500 to 6000.

The repeating unit of the heterobifunctional hydrophilic polymer molecule is in the range of 4 to 450, preferably 20 to 225, more preferably 30 to 130.

In the present invention, the heterobifunctional hydrophilic polymer molecule serves as a spacer in addition to the cross-linking agent. If the molecular weight is excessively low, the spacer function cannot be sufficiently brought out. If the molecular weight is excessively high, the quantity of the cross-linking agent used in the immobilization requiring a certain molar concentration thereof will be undesirable increased.

In order to inhibit non-specific absorption, the polymer molecule serving as the cross-linking agent in the present invention is required to be hydrophilic. The term “hydrophilic” or “hydrophilicity” herein means that the polymer molecule serving as a cross-linking is water-soluble or has water-solubility.

For example, the hydrophilic polymer molecule includes polyethylene glycol, polyvinyl alcohol, polyacrylic acid (or polymethacrylic acid), polyacrylate (or polymethacrylate), polyacrylamide (or polymethacrylamide), polyethyleneimine, polyvinylpyrrolidone, polyester or polyurethane copolymerized with a hydrophilic region of polyethylene glycol or monomer containing carboxylic acid or its salts or sulfonic acid or its salts, carboxymethylcellulose, dextran, and polysaccharide such as chitosan, carrageenan or glucomannan.

A non-ionic polymer molecule is preferable selected so as to inhibit the non-specific absorption through ionic bonding. Specifically, it is preferable to select polyethylene glycol (PEG), polyacrylamide (or polymethacrylamide) or polyvinylpyrrolidone, which has no region reactive to OH group, carboxylic acid or its salts, amine and imine.

Among them, it is preferably to select a polymer molecule having a structure expressed by a repeating unit: —(—O—R₁—)ₙ—, wherein R₁is an alkylene group having a carbon number of 2 to 5, and n is an integer number of 4 to 450.

R may specifically include a straight-chain alkylene group such as —(—CH₂—CH₂—O——or —(—CH₂—CH₂—CH₂—O—)—, and a branched-chain alkylene group such as —(—CH(CH₃)—CH₂—O—)—. R may also include a block copolymer such as —(—CH₂—CH₂—O—)ₚ—(—CH(CH(CH₃)—CH₂—O—)_{q}—.

Among them, in view of hydrophilicity and flexibility of the chains, it is preferable to select a polymer molecule having the repeating unit of ethylene glycol: —(—CH₂—CH₂—O—)—.

Process of Immobilizing Biomolecule

Without being limited thereto, a biomolecule may be immobilized using a cross-linking agent through a process comprising immobilizing a functional group X at one terminal of the cross-linking agent onto the surface through the reaction therebetween, and then immobilizing the biomolecule using another functional group Y at the opposite terminal.

More specifically, when a heterobifunctional hydrophilic polymer molecule having an amino group at one terminal thereof, and a carboxyl group at the other terminal is used as the cross-linking agent, a solid surface having a carboxyl group introduced thereon is activated using carboxyimide and N-hydroxysuccinimide, and then the cross-linking agent is immobilized onto the surface through the reaction with the amino group at the one terminal. Then, the biomolecule (A) is immobilized to the surface through the cross-linking agent using the carboxyl group introduced on the surface.

Otherwise, when a heterobifunctional hydrophilic polymer molecule having a succinimide group at one terminal thereof, and a maleimide group at the other terminal is used as the cross-linking agent, the succinimide group of the cross-linking agent is reacted with a solid surface having an amino group introduced thereon. Then, the biomolecule (A) is immobilized to the surface through the cross-linking agent using the maleimide group introduced on the surface.

The biomolecule (A) may be bonded to the heterobifunctional hydrophilic polymer molecule serving as the cross-linking agent, directly or indirectly through another intervening substance. The type of bonding includes, without being limited thereto, covalent bonding, ionic bonding, chelate bonding and hydrogen bonding.

In the present invention, after using the heterobifunctional cross-linking agent, a functional group or substance capable of bonding with the biomolecule may be additionally introduced to the heterobifunctional cross-linking agent. For example, a nitrilotriacetic acid (NTA) group is immobilized using the heterobifunctional cross-linking agent, and then the NTA group and histidine tag are chelate bonded together using Ni-chelate to introduce a histidine tag protein on the surface through the cross-linking agent. Alternatively, biotin or streptoavidin is introduced into the cross-linking agent through a reaction with the functional group Y of the cross-linking agent, and then the introduced biotin or streptoavidin is reacted with the biomolecule (A).

Preferably, a solid surface for immobilizing the biomolecule thereto is a flat substrate in view of suitability to interaction analysis.

The flat substrate may be a metal substrate suitable to the interaction analysis through the surface plasmon resonance (SPR) method.

The term “metal substrate” includes any substrate which has a surface of metal at least on one side. The whole body of the metal substrate may be made of metal, or the metal layer may be formed on nonmetallic material. The nonmetallic material may include glass, ceramics and plastics, and preferable nonmetallic material is a transparent material, like glass, transparent plastics (poly polycarbonate, polyethylene terephthalate, PMMA, polystyrene polymethyl pentene et al.). The metal for the metal substrate may include gold, silver, copper, aluminum and chromium. In particular, a transparent substrate having a surface formed of a thin gold layer is preferable for the SPR method.

One of the reasons for suitability of the surface formed of a thin gold layer is that it allows a functional group to be introduced onto the surface through gold-sulfur bonding.

In the process of introducing a functional group onto the surface through gold-sulfur bonding, the compound expressed by the aforementioned general formula X′—R′—Y′may be used to facilitate the introduction of the functional group.

In the present invention, a background area of the solid surface other than the area (immobilization area) having the biomolecules immobilized thereto is preferably covered by a hydrophilic polymer molecule.

The hydrophilic polymer molecule may include the polymer molecules to be used in the aforementioned cross-linking agent, preferably the non-ionic polymer molecules. Specifically, it may be polyethylene glycol (PEG), polyacrylamide (or polymethacrylamide) or polyvinylpyrrolidone, which has no region reactive to OH group, carboxylic acid or its salts, amine and imine. Among them, PEG is particularly preferable. Because PEG has high hydrophilicity but no reactive functional group, it exhibits an excellent effect of inhibiting non-specific absorption and providing a large contrast relative to the spot area.

Preferably, the molecular weight of the hydrophilic polymer molecule is 1000 or more. If the molecular weight is less than 1000, the surface of the background area cannot inhibit non-specific absorption due to insufficient hydrophilicity in some cases. The upper limit of the molecular weight is not necessarily limited to a specific value. However, a molecular weight greater than 2000 is likely to increase the viscosity of the solution containing the polymer molecule, and result in immobilization of the polymer molecule to the surface in its entangled state. In this case, the polymer molecule immobilized without covalent bonding can be gradually detached from the surface to cause undesirable change in the base line of the sensor.

The hydrophilic polymer molecule may be immobilized directly or indirectly onto the background area. In the direct immobilization onto a gold surface, a hydrophilic polymer molecule at the terminal of a thiol group may be immobilized onto the surface. In the indirect immobilization onto a gold surface, alkanethiol having a functional group at the terminal thereof may be immobilized onto the gold surface, and then the hydrophilic polymer molecule may be immobilized based on a functional group in the alkanethiol, as with the aforementioned process. Alternatively, the hydrophilic polymer molecule may be coated on the surface.

The immobilization and background areas may be separated through patterning or stamping. For example, a patterning using light irradiation may be performed as follows. The hydrophilic polymer molecule for the background area is first immobilized on the entire surface. Then, the immobilization area is irradiated with ultraviolet light using a mask or the like for preventing the ultraviolet light from irradiating the background area, so as to oxidize the gold-sulfur bonding by the ultraviolet light, and the surface is cleaned to remove the oxidized region and expose the gold surface. Then, the aforementioned alkanethiol can be newly bonded onto the gold surface to allow a functional group onto the immobilization area.

Preferably, the hydrophilic polymer molecule for the background area has a plurality of metal-binding functional groups bonded on the metal surface.

In this hydrophilic polymer molecule having a plurality of metal-binding functional groups, hydrophilic polymer molecule may directly have the metal-binding functional groups, or may be bonded with functional groups introduced on the metal surface as described above.

If the hydrophilic polymer molecule having the plurality of metal-binding functional groups per molecule is bonded with the metal surface, it will never be detached from the metal surface unless all bindings are simultaneously destroyed. This can provide enhanced contrast between the background area and the immobilization area, and reduced variation in signals from the background area.

The plurality of metal-binding functional groups simply mean 2 or more such groups, preferably 3 to 16, more preferably 3 to 10. If the number is three or more, the possibility of detachment from the metal surface will be desirably reduced. However, if the number is seventeen or more, the number of the metal-binding functional groups freely residing without bonding with the metal surface is undesirably increased. For example, thiol groups selected as the metal-binding functional groups undesirably cause difficulty in immobilizing biomolecules having thiol groups by use of the cross-linking agent, due to the existence of a number of free thiol groups.

Preferably, the hydrophilic polymer molecule having the plurality of metal-binding functional groups has branched chains. More preferably, the metal-binding functional groups reside at branched chains. If the metal-binding functional groups are included in the main chain, the hydrophilic polymer will lay down on the surface, resulting in undesirably deteriorated ability of inhibiting non-specific absorption.

The metal-binding functional group is preferably a functional group including sulfur, particularly a thiol group or disulfide group. These functional groups are suitable to absorption bonding, particularly to a gold substrate. Thus, in this case, it is desired to use a gold substrate.

Preferably, in the immobilization area, any remaining functional groups other than those bonded with the double-stranded oligonucleotides are blocked. While any suitable blocking agent capable of reacting with the functional group and having no reactive functional group as described above may be used, the blocking agent is preferably a hydrophilic polymer molecule. This hydrophilic polymer molecule may include the aforementioned polymer molecules for use in the background area. Among them, PEG is particularly preferable.

It is understood that the hydrophilic polymer molecule serving as the blocking agent is required to have a functional group reactive to the remaining function groups in the immobilization area. Preferably, the functional group of the hydrophilic polymer molecule includes amine group and thiol group.

In the hydrophilic polymer molecule serving as the blocking agent, the functional groups are preferably located at the terminals of the polymer molecule, more preferably at one of the terminals. If the functional groups are located at the main chain of the polymer molecule or at a number of side chains branched from the main chain, they are likely to cause undesirable steric hindrance to an immobilized target molecule. Further, if a large number of the functional groups are located at the main chain or side chains, it is undeniable that even after the blocking reaction with the remaining functional groups in the immobilization area, some functional groups will be left in the polymer molecule, resulting in occurrence of non-specific absorption. Thus, the functional groups are located preferably only at the terminals. More preferably, the functional groups are located only at one of the terminals but not at the other terminal. Otherwise, the hydrophilic polymer molecule may include a methoxy group or hydroxyl group which has low activity.

The molecular weight of the hydrophilic polymer molecule is preferably 400 or more, more preferably 1000 or more. The reason is that the effect of inhibiting non-specific absorption is enhanced as the molecular weight/repeating unit is increased. However, a molecular weight of 50000 or more is likely to cause detrimental steric hindrance to an immobilized target molecule.

The above blocking technique can be applied to any other biochip having another kind of biomolecule bonded thereto, as well as double-stranded oligonucleotides.

Preferably, the biochip is provided with a marker indicative of spot positions in the array area. The marker makes it possible to readily identify where a substance immobilized to the array is located. Particularly, in the SPR imaging to be used as an optical detection method, images are typically picked up from the side of the back surface of the chip using a CCD camera. The image pickup from the back surface side provides a reversed image of the pattern on the chip, which leads to difficulty in identifying the position of a target spot. The marker allows the position of an intended spot to be identified without any difficulties.

Any suitable number of markers, i.e., one or more, may be provided. While the marker may have any suitable shape, it is preferably characters and/or numerals to facilitate identifying the row and column of the array. For example, if a commercially available 96-hole plate or 384-hole plate is used for preparing a sample for immobilizing biomolecules thereto, the markers may be designed such that the vertical and horizontal axes are discriminated using alphabetical characters A to H and numerals 1 to 12, respectively.

The marker may be introduced to the biochip through any suitable process allowing the marker to be detected by a certain detect system. For example, the process may include depositing metal on the surface of the chip except for the marker region, depositing metal on the surface of the chip to provide a different thickness only in the marker region, attaching a polymer, organic substance or inorganic substance, to any other region than the marker region of the metal surface or between the metal surface layer and substrate, and differentiating the substances to be attached to the marker region and the remaining region.

The polymer, organic substance or inorganic substance may be attached to the marker region or the remaining region by means of vapor deposition using a mask, coating using a mask, or printing such as letterpress or inkjetting. Alternatively, it may be attached through a process of coating the substance over the whole area of the metal surface and then decomposing/removing or transforming a substance attached at an unnecessary region by irradiating the region with light/laser/radiation, and optionally attaching another substance to the region. The above processes may be used individually or in combination. If the marker is attached onto a metal layer formed on the substrate, the marker can be attached onto the metal layer through the above process. If the marker is attached between the substrate and the metal layer, after attaching the marker onto the substrate through the above process, the metal layer may be formed on the substrate.

The polymer or organic substance may be selected from substances capable of being attached to the metal layer or the substrate, and of being retained in a proper position during supply of a measurement liquid thereto. For example, the polymer or organic substance includes a polymer or organic for use in conventional ink or paint, preferably a polymer or organic containing sulfur, such as thiol, sulfide or disulfide, in its molecule capable of firmly bonding with gold.

The marker is formed on the surface of the tip to limit the difference in height relative to another area to 3 μm or less, preferably 1 μm or less, more preferably 100 nm or less, even more preferably 50 nm or less. Further, the marker is preferably introduced in a monomolecular layer. In an operation of exposing the tip surface to a solution containing a target substance to be analyzed, a tip surface with irregularity of greater than 3 μm undesirably hinders the flow of the solution and adversely affects the evaluation of interaction kinetics. Thus, it is desired to limit the irregularity to within the biomolecular level. Even if the irregularity is set at the biomolecular level, it can be sufficiently identified through an optical detection method such as the SPR imaging.

The irregularity of the surface may be measured using a tracer-type or no-contact-type surface-roughness meter, an interference microscope, a scanning tunneling microscope or an SPR apparatus (calculation from SPR angle, observation of tomographic image). The measurement method or apparatus may be appropriately selected depending on the magnitude of the irregularity.

Preferably, the markers are formed in conjunction with the formation of the spots. If either ones of markers and spots are formed in advance of the other, it is likely to cause restricted pattern of the spots or displacement between the markers and the spots. The term “in conjunction with” herein means not only forming markers and spots concurrently but also forming markers and spots in the same step or continuously without moving the substrate. Specifically, markers may be formed in conjunction with the formation of spots through a process of forming markers and spots by use of a mask during the formation of the metal layer through vapor deposition or the like, a process of forming markers during a spotting operation for forming spots on the metal surface, or a process of forming markers during an operation for subjecting a portion of the metal surface corresponding to the background area to surface treatment so as to form spots.

In the process of forming markers during a spotting operation for forming spots on the metal surface, the spots may be formed on the metal surface by an automatic spotter or the like, and the markers are formed during this spotting operation using the same substance as or a different substance from that of the spots. The spots and markers may be formed through a method using a pen or pin, or inkjetting. Alternatively, the markers may be engraved on the substrate by use of laser or the like after completion of the spotting operation.

In the process of forming markers during an operation for subjecting a portion of the metal surface corresponding to the background area to surface treatment so as to form spots, when a substance, such as hydrophilic compound, for the background area is coated on the whole area of the metal surface, and then the substance corresponding to the spots is partially removed, the substance corresponding to the markers may also be removed.

The biomolecule (A) is not limited to the aforementioned double-stranded oligonucleotide, but any other suitable biomolecule, such as nucleic acid, protein, peptide or sugar chain, may be used. Among them, nucleic acid is preferably selected in view of its stability and variety in commercially available types.

The biomolecule (A) may be a single substance or a plurality of different substances. The use of a plurality of different substances allows high-throughput analysis. Through high-throughput analysis, a number of compounds can be efficiently evaluated. The number of the different kinds of the substances or biomolecules is eight or more, preferably ten or more, more preferably twelve or more, even more preferably twenty or more. The upper limit of the number is typically about ninety-six, without being limited thereto. Preferably, a plurality of substances are immobilized in an array arrangement. This arrangement allows biomolecule interaction to be analyzed at each of the positions where the biomolecules are immobilized.

The biomolecule (A) immobilized according to the present invention has excellent mobility, and non-specific absorption is inhibited so as to allow the interaction with the biomolecule (B) or aggregate thereof to be measured under adequate conditions.

The biomolecule (B) is not limited to a specific kind, but it may include nucleic acid, protein, peptide and sugar chain. Further, an aggregate of the biomolecules (B) such as protein dimer may be used.

The present invention is suitable in particular for the measurement of a protein having a complex structure such as transfer factors. Most of the transfer factors have a particular 3-dimensional structure as in b-zip or zinc finger, and cannot readily access a target nucleic-acid molecule to be bonded, if the target molecule has insufficient mobility. In addition, due to extremely high non-specific absorption to a surface, the transfer factors can be analyzed only if non-specific absorption is adequately inhibited. The present invention, which is capable of ensuring the mobility of the immobilized molecule and inhibiting non-specific absorption, can be effectively used in the analysis for evaluating the interaction with such transfer factors having a particular structure.

According to the method of the present invention, the interaction with the aggregate of the biomolecules (B) can also be adequately analyzed. Thus, the method of the present invention can be used to achieve extremely significant measurements, such as evaluation of the difference in bonding behavior between various combinations of heterodimers.

The interaction measurable using the present invention may include protein-protein interaction, nucleic acid-protein interaction, nucleic acid-nucleic acid interaction, protein-peptide interaction, protein-carbohydrate chain interaction and antigen-antibody interaction.

Measurement Method

The surface plasmon resonance (SPR) method is preferably used to measure the interaction between the immobilized biomolecule (A) and the biomolecule (B) or an aggregate thereof. As the surface plasmon resonance (SPR) method, SPR imaging is particularly preferable.

The SPR method allows a label-free and real-time measurement. Thus, the SPR method is particularly useful in case that at least one of the biomolecules (A) and (B) is a protein.

In the measurement based on SPR, a contact liquid located between a transparent substrate and a prism preferably has a low variation in weight due to vaporization. Specifically, the weight decrease rate after being maintained in an uncontrolled state at 37° C. for 1 hour is preferably 1% or less, more preferably 0.5% or less. A weight decrease rate of greater than 3% is likely to cause the occurrence of interference patterns.

The weight decrease rate of the contact liquid is measured as follows (the details of this measurement will be described in connection with Examples).

1. 200 μl of contact liquid is obtained, and fed into a tubular weighing bottle (available from AS ONE Co.) having a body diameter of 18 mm (inner diameter: 16 mm) and a height of 35 mm.

2. The weighting bottle containing the contact liquid is put in a temperature/humidity-controlled room having a space of 0.5 m³ or more, and left therein at a temperature of 37±0.5° C. and a humidity of 50% RH or less for 1 hour under the condition that a cap of the weighting bottle is opened and the weighting bottle is not directly exposed to strong airflow, to measure the change in weight of the contact liquid due to vaporization.

The refractive index (n_{d}) of the contact liquid is preferably 1.60 or more, more preferably 1.65 or more. A higher refractive index allows an incident light angle for measuring SPR to be reduced so as to facilitate the measurement. Particularly in the SPR imaging, the higher refractive index can reduce the distortion in the uniaxial direction of an image. Thus, it is desired to use a contact liquid having a high refractive index.

The boiling point of the contact liquid is preferably 200° C. or more. A contact liquid having a boiling point of less than 200° C. exudes a smell indicative of some volatility. Any vaporization of the contact liquid during measurement is highly likely to adversely affect the measurement.

Specifically, instead of diiodomethane having a boiling point of 181° C., it is preferable to use 1-bromonaphthalene having a boiling point of 279° C., or 1-iodonaphthalene having a higher boiling point. 1-bromonaphthalene and 1-iodonaphthalene can be mixed to prepare a contact liquid having a refractive index of up to n_{d}=1.70.

Such a contact liquid having a high boiling point and reduced volatility can advantageously eliminate most of the interference pattern caused by variation in refractive index due to vaporization of a contact liquid so as to achieve an accurate SPR measurement.

### EXAMPLE

The present invention will be specifically described below in connection with Inventive Examples and Referential Examples. Those skilled in the art will recognize that these Examples are not intended to limit the present invention.

### Inventive Example 1

Through vapor deposition, a chromium layer having a thickness of 3 nm was formed on a SF 10 transparent glass substrate of 18 mm square and 1 mm thickness, and then a gold layer having a thickness of 45 nm was formed on the chromium layer. The thickness of the deposited layer was monitored using a crystal oscillator. The substrate with a surface having the deposited gold layer was immersed in 1 mM ethanol solution of 8-amino-1-octanethiol, hydrochloride (8-AOT, available from Dojindo Laboratories) for 16 hours to form a self-assembled 8-AOT on the gold surface of the substrate. Heterobifunctional polyethylene glycol (NHS-PEG-MAL, available from Shearwater Polymers Inc.) having a molecular weight of 3400 and a terminal with a succinimide (NHS) group and a maleimide (MAL) group was dissolved at 10 mg/l in a phosphoric acid buffer solution (20 mM phosphoric acid, 150 mM NaCl, pH 7.2), and reacted with the 8-AOT on the gold surface for 2 hours. As a result of the reaction between an amino group of the 8-AOT and the NHS group of NHS-PEG-MAL, the MAL group remaining in unreacted state could be introduced onto the metal surface through PEG.

Two kinds of 5′ thiol-terminated DNAs were hybridized, and the obtained double-stranded DNAs (dsDNAs) were spotted onto the above surface using a HandSpotter of Greiner Bio-One GmbH. The surface and the dsDNAs were reacted with one another as in uncontrolled state for 15 hours to immobilize the dsDNAs on the surface.

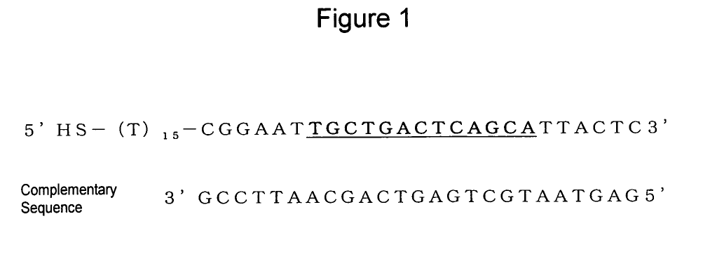

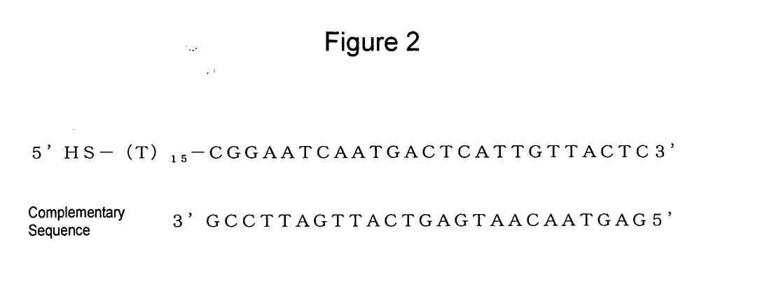

The immobilized DNAs are a Maf recognition sequence (MARE 25) and a Maf non-recognition sequence (MARE 23), their details of which are shown in FIGS. 2 and 1, respectively. Each of the sequences is designed such that a purposive sequence is linked to a 5′ thiol terminal through a thymine 15-base spacer.

Each of the DNAs were hybridized by adding a thiol-terminated DNA and a DNA complementary to the thiol-terminated DNA into a 5×SSC solution (75 mM sodium acid citrate, 750 mM NaCl, pH 7.0) to prepare the mixture containing 25 μM of the thiol-terminated DNA and 100 μM of the complementary DNA, heating the mixture in boiling water for 5 minutes, rapidly cooling and maintaining the mixture at 0° C. for 15 minutes, and then incubating the mixture at 37° C. for 3 hours.

After the surface with the immobilized dsDNAs was rinsed with a phosphoric acid buffer solution, the substrate was set up in an SPR imaging apparatus (SPR Imager, available from GWC Instruments Inc.), and a transfer-factor-measuring buffer solution (10 mM Hepes, 300 mM NaCl, 4 mM MgCl, 1 mM EDTA, 100 μml cow serum albumin, pH 7.9) was supplied to a flow cell.

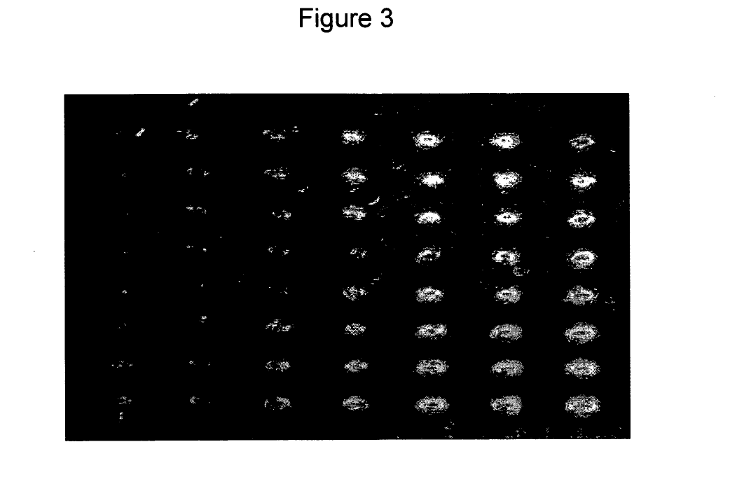

FIG. 3 shows an image obtained by the SPR imaging. The elliptical region having the immobilized dsDNA is observed whitishly due to the change of refractive index. As shown in FIG. a plural number of the dsDNAs are immobilized on the surface in an array arrangement.

After a signal from the SPR imaging apparatus became stable, a solution prepared by dissolving a homodimer of transfer factor MafG in the above transfer-factor-measuring buffer solution at a concentration of 1 μml was supplied to the cell at a rate of 100 μl/min for 10 minutes, and then the buffer solution containing no transfer factor was supplied to the cell to observe the change in the SPR signal representing association/dissociation.

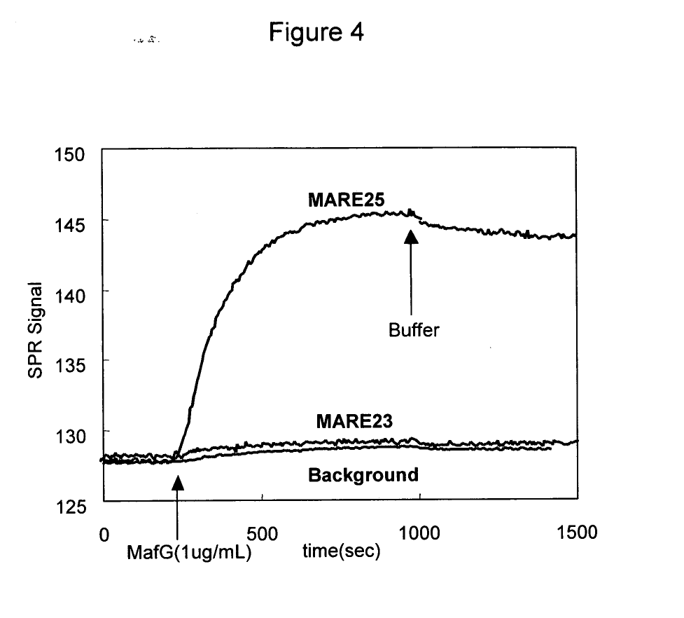

The observation was performed at three points: two regions each having the immobilized MARE 25 or MARE 23, and one region having no DNA (background position). FIG. 4 shows the change of the SPR signal. It can be observed that the MafG homodimer is bonded only to the sequence of MARE 25, and substantially no MafG homodimer is bonded to MARE and the background position. An association rate constant, a dissociation rate constant and an association equilibrium constant were 2.22×10⁵(M⁻¹s⁻¹), 8.80×10⁻⁴(s⁻¹) and 2.52×10⁸(M⁻¹), respectively.

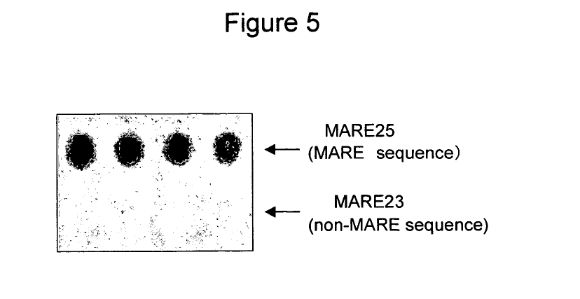

The difference between one image picked up before supplying the MafG homodimer into the cell, and another image picked up ten minutes after completion of the supply was evaluated by processing these images using an image processing software NIT Image. FIG. 5 shows this result. As seen in FIG. 5, the MafG homodimer is bonded only to the sequence of MARE 25.

### Referential Example 1

As with Inventive Example 1, an 8-AOT self-assembled surface was formed on a gold-deposited transparent substrate, and then a heterobifunctional hydrophilic polymer molecule NHS-PEG-MAL having a succinimide (NHS) group and a maleimide (MAL) group was immobilized onto the surface.

Then, respective single-stranded DNAs of MARE 25 sequence and MARE 23 sequences each having thiol at its 5′ terminal were dissolved in a 1 mM phosphoric acid buffer solution. Then, the mixture was spotted on the surface in an array arrangement to immobilize the single-stranded DNAs on the surface through reaction with the surface for 15 hours.

After the surface with the immobilized DNAs was rinsed with a phosphoric acid buffer solution, the substrate was set up in the SPR imaging apparatus, and the flow cell was filled with the transfer-factor-measuring buffer solution. Then, a DNA complementary to MARE 25 was added into the cell at a concentration of 1 μM, and left for 20 minutes to hybridize the complementary DNA to MARE 25. After rinsing the surface, a DNA complementary to MARE 23 was added into the cell at a concentration of 1 μM, and left for 20 minutes to hybridize the complementary DNA to MARE 23.

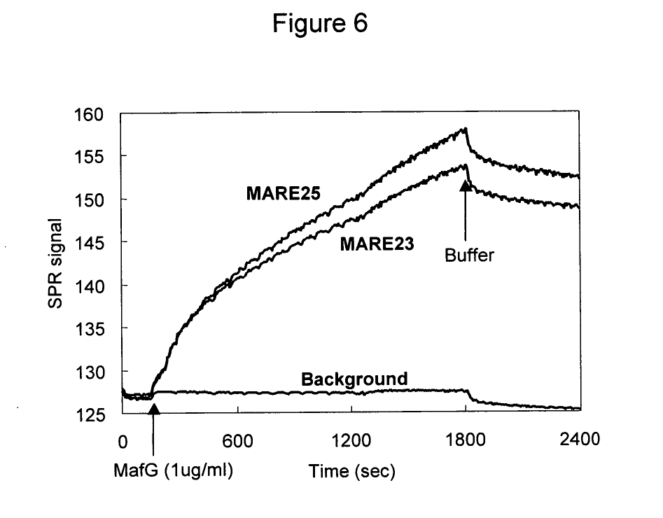

The interaction with the MafG homodimer was observed through the same process as in Inventive Example 1. As a result, the MafG homodimer was absorbed to both MARE 25 and MARE 23, and no specificity of the transfer factor MafG could be observed (FIG. 6). This would be caused because the DNA complementary to MARE 25 was also bonded to MARE 23 to preclude the formation of the intended double-stranded DNA.

### Inventive Example 2

4-arm PEG (SUNBRIGHT PRE-100SH, available from NOF Co.) having a thiol group as a terminal function group was dissolved in 7 ml of mixed solution of ethanol:water=6:1, at a concentration of 1 mM. The 4-arm PEG has high hydrophilicity because it is a molecule which has a molecular weight of 10000 and includes four PEG chains each having approximately the same length from the center. The 4-arm PEG also exhibits metal-binding activities because all of the terminals of the PEG have a thiol group.

Through vapor deposition, a chromium layer having a thickness of 3 nm was formed on a SF 15 glass slide of 18 mm square and 2 mm thickness, and then a gold layer having a thickness of 45 nm was formed on the chromium layer. The obtained gold-deposited slide was immersed in the above 4-arm PEG thiol solution for 3 hours to bond the 4-arm PEG thiol solution onto the whole area of the gold substrate.

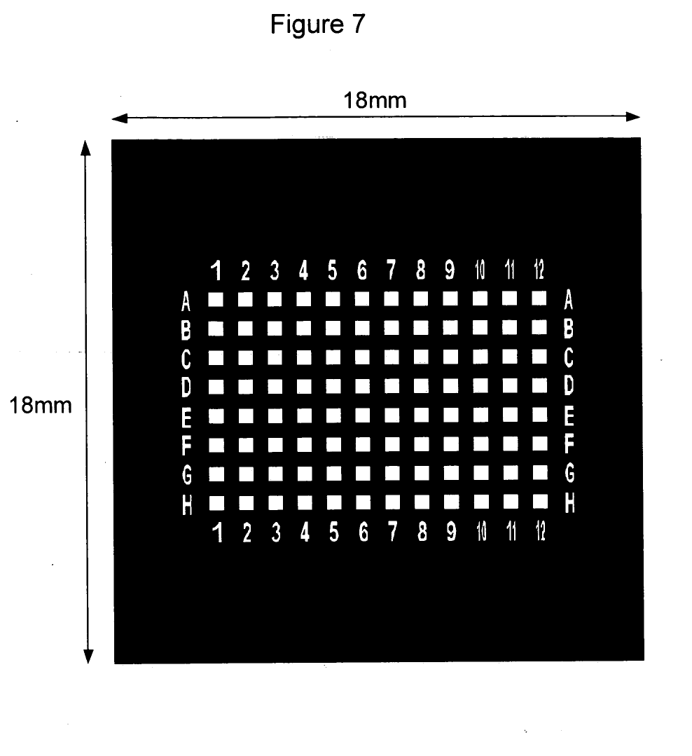

A photomask as shown in FIG. 7 was placed on the slide. Then, the slide was irradiated with light from a 500 W ultra-high pressure mercury lamp (available from Ushio Inc) for two hours to remove the 4-arm PEG thiol at regions irradiated with UV. The photomask was formed with ninety-six holes of 5 mm square and 1 mm intervals. The holes of the photomask allow UV light to pass through so that regions of the slide corresponding to the holes are irradiated with the UV light and patterned. The 4-arm PEG at the remaining region irradiated with no UV light is left to serve as a background area or reference area. The photomask is also formed with character-shaped and numerical-shaped holes for forming on the slide markers which indicate the respective positions of spots and correspond to respective marks of samples prepared on a 96-hole plate.

The slide is immersed in 1 mM ethanol solution of 8-AOT for 1 hours to form a self-assembled 8-AOT surface on the UV-irradiated regions of the slide. Heterobifunctional polyethylene glycol (NHS-PEG-MAL, available from Nektar) having a molecular weight of 3400 and a terminal with a succinimide (NHS) group and a maleimide (MAL) group was dissolved at 10 mg/l in a phosphoric acid buffer solution (20 mM phosphoric acid, 150 mM NaCl, pH 7.2), and reacted with the 8-AOT on the gold surface of the slide for 2 hours. As a result of the reaction between an amino group of the 8-AOT and the NHS group of NHS-PEG-MAL, the MAL group remaining in unreacted state could be introduced onto the metal surface through PEG.

Two kinds of DNAs were hybridized, and spotted onto the obtained surface using an automatic spotter (MultiSPRinter™ Spotter, available from Toyobo Co., Ltd.) to form spots each having the two kinds of double-stranded DNAs (dsDNAs) immobilized thereto, and spots (blank) having no DNA. The respective sequences of the above DNAs are shown in Table 1. In each of the dsDNAs, the anchor-side DNA is designed such that a purposive sequence is linked to a 5′ thiol terminal through a thymine 15-base spacer. The complementary-side DNA has only a sequence complementary to the purposive sequence of the anchor-side DNA, but not the thiol and the thymine 15-base spacer. In Table 1, GATA reg is a sequence capable of being recognized by and bonded with GATA-1, wherein a GATA sequence is included at the center relative to the 5′ terminal. GATA mut is a sequence in which GATA in GATA reg is substituted with AGTA, or G and A in GATA reg are reversed, and generally incapable of being recognized by GATA-1.

TABLE 1

| | |
|---|---|
| Sequence | |
| Name | Sequence (5′→3′) |

| | | | |
|---|---|---|---|
| GATA reg | anchor-side | HS-(T)₁₅- | (SEQ ID NO: 1) |
| | | CGGAATGATAATTACG | |
| | complemen- | CGTAATTATCATTCCG | (SEQ ID NO: 2) |
| | tary-side | | |
| GATA mut | anchor-side | HS-(T)₁₅- | (SEQ ID NO: 3) |
| | | CGGAATAGTAATTACG | |
| | complemen- | CGTAATTATCATTCCG | (SEQ ID NO: 4) |
| | tary-side | | |

The maleimide group introduced in the surface of the chip reacted with the thiol group of the anchor-side DNA to allow the dsDNA to be immobilized on the surface by covalent bonding.

The hybridization of the DNAs was performed under the same conditions as those in Inventive Example 1. The obtained dsDNAs were spotted each at about 1 nl, and immobilized on the surface through the reaction with the surface for 15 hours.

Blocking of Unreacted Maleimide Group

In order to block the unreacted maleimide group, after the surface with the immobilized dsDNAs was rinsed with a phosphoric acid buffer solution, PEG thiol (SUNBRIGHT MESH-50H, available from NOF Co.) having a thiol group as a function group at one terminal and a methoxy group as a functional group at the other terminal was dissolved in a phosphoric acid buffer solution (20 mM phosphoric acid, 150 mM NaCl, pH 7.2) at a concentration of 10 mg/ml. Then, 250 μl of the obtained mixture was coated on the chip, and reacted for 1 hour. The molecular weight of the PEG thiol is 5000, which means extremely high hydrophilicity capable of effectively inhibiting non-specific absorption.

Measurement Using SPR Imaging

The obtained dsDNA-immobilized tip was set up in an SPR imaging apparatus (MultiSPRinter™, available from Toyobo Co., Ltd.), and a transfer-factor-measuring buffer solution (20 mM Hepes, 300 mM NaCl, 0.2 mM ZnCl₂, 0.005% Tween 20, pH 7.9) was supplied to a flow cell.

After a signal from the SPR imaging apparatus became stable, a solution prepared by dissolving a transfer factor GATA-1 in the above transfer-factor-measuring buffer solution at a concentration of 10 μM was supplied to the cell in the SPR apparatus. In the supply of the GATA-1, polydIdC was added to the buffer solution at a concentration of 100 μg/ml to inhibit non-specific absorption to nucleic acid. The solution was supplied to the cell at a rate of 100 μl/min for 10 minutes, and then the buffer solution containing no transfer factor was supplied to the cell to observe the change in the SPR signal representing association/dissociation.

The observation of the change in the signal was performed at four regions: GATA reg, GATA mug, Blank and Background.

Observation Result & Analysis

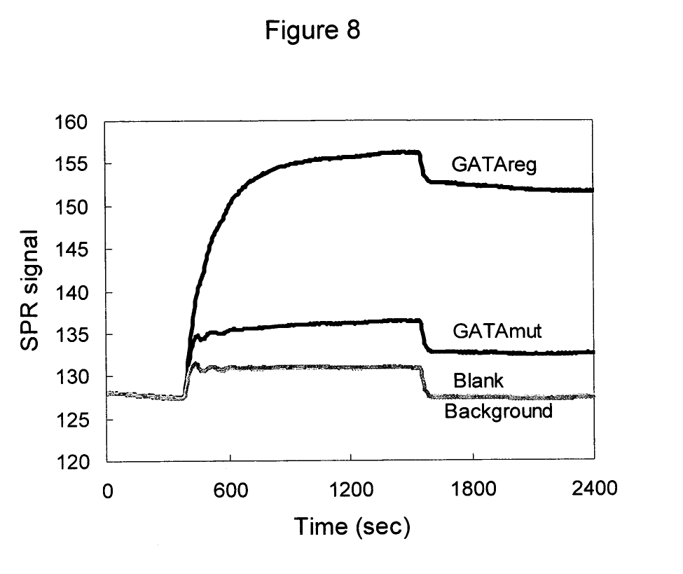

FIG. 8 shows the change in the signal. It can be observed that GATA-1 is bonded to the sequence of GATA reg, and substantially no GATA-1 is bonded to the sequence of GATA mut. In FIG. 8, no signal change is observed in the background region, which confirms that substantially no GATA-1 is bonded to the background region.

### Referential Example 2

### Demonstration on Effect of Plurality of Metal-Binding Groups

A glass slide having the same pattern as that in Inventive Example 2 was immersed in a solution of 1 mM 7-carboxy-heptanethiol (7-CHT, available from Dojindo Laboratories) for 2 hours to introduce an amino group to UV-irradiated regions. Further, it was determined whether 4-arm PEG was exchanged with 7-CHT.

In order to determine whether the exchange reaction was caused, the chip was set up in a surface plasmon resonance (SPR) imaging apparatus (available from Toyobo Co., Ltd.) to observe the absorption of poly-L-lysine. The measurement was performed using a 10 mM phosphoric acid buffer solution (150 mM NaCl, pH 7.4, 30° C.). Poly-L-lysine (available from Sigma-Aldrich Co.) having a molecular weight of 4000-15000 was dissolved in the above buffer solution, and the obtained solution was kept in contact with the chip for 5 minutes. The poly-L-lysine is a polymer having positive charges and exhibiting characteristics of electrostatically bonding to a region where a carboxyl group is introduced through 7-CHT bonded thereto.

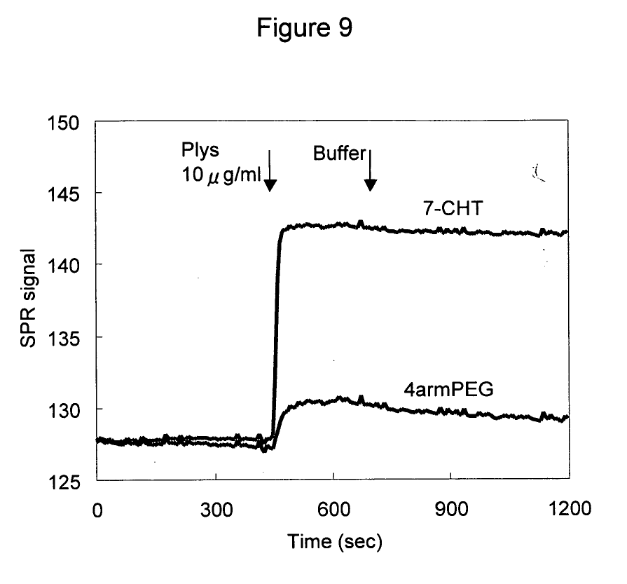

FIG. 9 shows the change in the SPR signal. The change in the signal level according to the poly-L-lysine between before the supply of the poly-L-lysine and after 5 minutes from the completion of the supply was measured. The signal in the region having 7-CHT introduced thereto was obtained by averaging the signals in 96 of the 7-CHT-introduced regions. The signal of the 4-arm PEG region was obtained by averaging signals in a vertically elongated oblong area consisting of 11 regions extending in the column direction of the array between the spots. The signal according to the poly-L-lysine in the 7-CHT-introduced region was 13.9. In contrast, the signal according to the poly-L-lysine in the 4-arm-PEG-bonded region was 1.5, or the signal ratio was 9.1. This means that substantially no exchange reaction of 7-CHT was caused in the 4-arm-PEG-bonded region. This would be caused from the fact that, while 4-arm PEG has no long alkyl chain, it is bonded with gold at a plurality of positions, and thus substantially no disassociation occurs.

In this manner, a biochip with an array having hydrophilic polymer molecules free from disassociation could be readily obtained. In this biochip, a biomolecule can be bonded thereto using, as a starting or initial point, the carboxyl group of 7-CHT introduced only in the UV-irradiated regions. The 4-arm-PEG-bonded regions having substantially no functional group allows non-specific absorption to be effectively inhibited.

### Referential Example 3—Comparison with Referential Example 2

The same test was performed under the same conditions as those in Referential Example 2 except that 4-arm-PEG was replaced by PEG thiol (SUNBRIGHT MESH-50H, available from NOF Co.) having only one thiol group per molecule. The molecular weight of the PEG thiol is 5000, which means extremely high hydrophilicity. As described above, the PEG thiol has one terminal of a thiol group exhibiting metal binding activities, and the other terminal of a methoxy group. The carbon number of the alkyl chain site in the PEG thiol is two, which means relatively low hydrophobic binding activities between molecules.

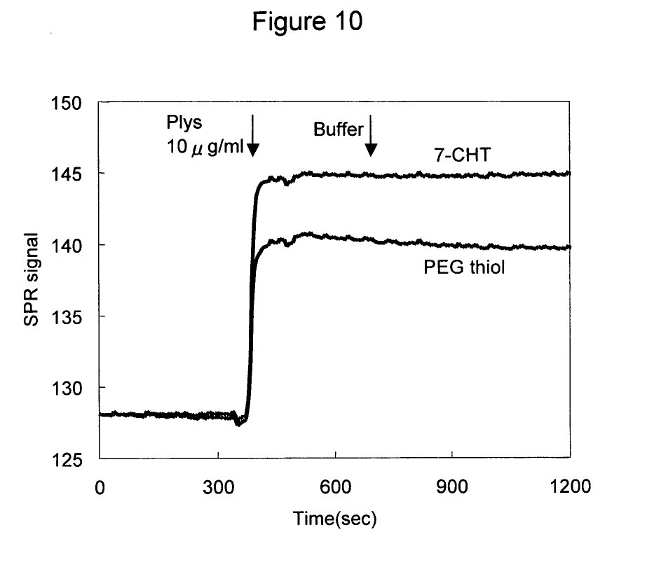

FIG. 10 shows the change in the SPR signal. The signal in the 7-CHT-introduced region (UV-irradiated region) was 16.9. In contrast, the signal according to poly (ethylene imine) (PEI) in the PEG thiol-bonded region was 11.9, or the signal ratio was 1.4. This means that the exchange reaction of 7-CHT was caused in the PEG-thiol-bonded region, and a carboxyl group also exists in the PEG region. This would be caused by the fact that, while PEG thiol has no long alkyl chain, it has only one metal-binding functional group, and thus disassociation from the gold surface was caused easily. That is, the PEG thiol region is unstable as compared to Referential Example 2, and a biomolecule can also be bonded to the PEG thiol region. Further, it would have higher possibility of electrostatic non-specific absorption.

### Inventive Example 3

As with Inventive Example 2, a maleimide group was introduced to a gold surface of a slide glass through PEG.

Six kinds of DNAs were annealed, and spotted onto the obtained surface using an automatic spotter (MultiSPRinter™ Spotter, available from Toyobo Co., Ltd.) to form spots each having the two kinds of double-stranded DNAs (dsDNAs) immobilized thereto, and spots (blank) having no DNA. In each of the dsDNAs, the anchor-side DNA is designed as 5′ HS-(T)₁₅-CGGAAT (N)₁₃TTACTC 3′ (SEQ ID NO:5), or a purposive sequence is interposed in the 13-base site of (N)_{13.} The six kinds of respective purposive sequences are shown in Table 2. While the sequence of the complementary-side DNA is not shown in Table 2, complementary chains corresponding to the 25-base of CGGAAT (N)₁₃TTACTC are prepared in each sequence.

TABLE 2

5′ 3′

| | | |
|---|---|---|
| MARE 25 | T G C T G A C T C A G C A | (SEQ ID NO: 6) |
| hOPSIN | T G C T G A T T C A G C C | (SEQ ID NO: 7) |
| HNQO1m | A G T T G A C T C A G C A | (SEQ ID NO: 8) |
| MARE23 | C A A T G A C T C A T T G | (SEQ ID NO: 9) |
| hBglHS4 | G G C T G A C T C A C T C | (SEQ ID NO: 10) |
| mGSTY | T G G T G A C A A A G C A | (SEQ ID NO: 11) |

The maleimide group introduced in the surface of the chip reacted with the thiol group of the anchor-side DNA to allow the dsDNA to be immobilized on the surface in the form of covalent bonding.

The hybridization of the DNAs was performed under the same conditions as those in Inventive Example 1. The obtained dsDNAs were spotted each at about 1 nl, and immobilized on the surface through the reaction with the surface for 15 hours.

Blocking of Unreacted Maleimide Group

The same operations as in Inventive Example 2 were performed.

Measurement Using SPR Imaging

The obtained dsDNA-immobilized tip was set up in an SPR imaging apparatus (MultiSPRinter™, available from Toyobo Co., Ltd.). During setting operation, the back surface of the tip (glass surface) was brought into contact with an SF15-60° prism through matching oil. Cargill B-series (nd=1.700, available from Cargill Inc.) having extremely low volatility was used as the matching oil. This oil is characterized by a weight change of 0.11%, or 1% or less, under the condition of leaving it exposed to the atmosphere at 37° C. for 1 hour, and a boiling point of 279° C., or 200° C. or less.

After setting up the tip in the apparatus, a transfer-factor-measuring buffer solution (20 mM Hepes, 200 mM NaCl, 4 mM MgCl₂, 1 mM EDTA, 11 μg/ml BSA, pH 7.9) was supplied to a flow cell.

After a signal from the SPR imaging apparatus became stable, a solution prepared by dissolving a transfer factor MafG in the above transfer-factor-measuring buffer solution at a concentration of 125 μM was supplied to the cell in the SPR apparatus. The solution was supplied to the cell at a rate of 100 μl/min for 10 minutes, and then the buffer solution containing no transfer factor was supplied to the cell to observe the change in the SPR signal representing association/dissociation.

The observation of the signal change was performed at regions of the six kinds of gene sequences, B1 ank and Background.

Observation Result & Analysis

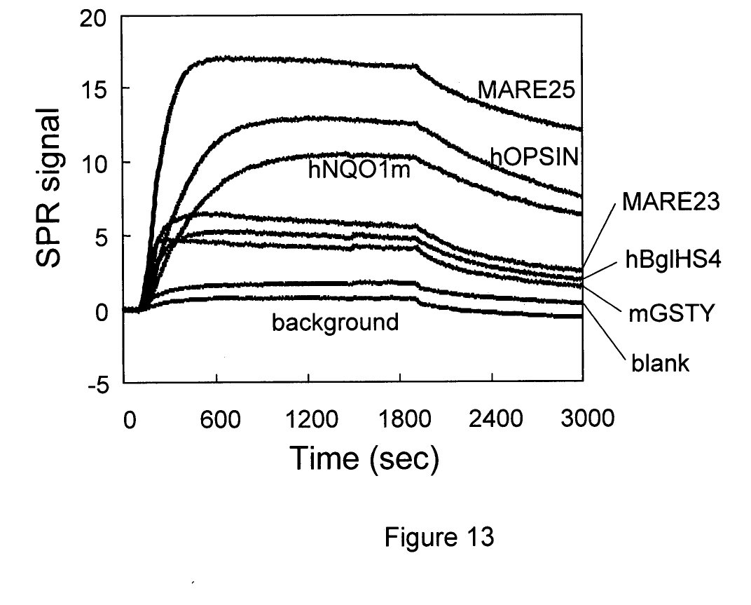

FIG. 13 is a graph showing the signal change. In this manner, the interactions at six points on a single chip could be simultaneously observed. In this example, it could be confirmed that the bonding strength to MARE 25 as a consensus sequence is high, and the bonding strength to MARE 23 is low. As seen in FIG. 13, no signal change is observed in the background region (4-arm PEG), which shows that substantially no MafG was bonded thereto. The comparison between the kinetics value obtained from a association/disassociation curve and the value obtained through a gel mobility shift assay (GMSA) as a conventional interaction-measuring method is shown in the following table. While there are some differences between the respective values of SPR and GMSA, the bonding strength could be sufficiently evaluated through the SPR in view of affinity and bonding strength.

TABLE 3

| | SPR average | | | |
|---|---|---|---|---|
| | ka[m⁻¹s⁻¹]10⁵ | Kd[S⁻¹]10⁻⁴ | K_{D}[M]10⁻⁹ | GMSA K_{D}[M]10⁻⁷ |
| MARE 25 | 1.36 +/− 0.32 | 3.13 +/− 0.66 | 2.50 +/− 1.02 | 2.49 +/− 0.66 |
| hOPSIN | 0.493 +/− 0.062 | 3.72 +/− 0.03 | 7.67 +/− 1.17 | 2.52 +/− 0.05 |
| hNQO1m | 0.385 +/− 0.035 | 3.24 +/− 0.36 | 8.58 +/− 1.75 | 2.73 +/− 0.13 |
| mGSTY | N.D | N.D | N.D | N.D |
| MARE23 | N.D | N.D | N.D | N.D |
| hBglHS4 | N.D | N.D | N.D | N.D |

As mentioned above, the double-stranded oligonucleotides according to the present invention can be used to adequately analyze the interaction between oligonucleotides and biomolecules or aggregates thereof.

The method according to the present invention can prepare double-stranded oligonucleotides suitable for observation of biomolecule interactions efficiently and adequately.

The present invention provides highly useful means for use in measuring the interaction between oligonucleotides and biomolecule or aggregate thereof.

Furthermore, the present invention makes it possible to immobilize biomolecules while maintaining their mobility and activities/functions. The method according to the present invention also makes it possible to evaluate the interaction kinetics between one biomolecule and another biomolecule or aggregate thereof while inhibiting non-specific absorption.

What is claimed is:

1. An array comprising a plurality of double-stranded oligonucleotides immobilized on a metal substrate, each of said double-stranded oligonucleotides including a first single-stranded oligonucleotide and a second single-stranded oligonucleotide, said first and second single-stranded oligonucleotides being entirely or partially bonded together in a complementary manner to form said double-stranded oligonucleotide, wherein among said first and second single-stranded oligonucleotides, only said first single-stranded oligonucleotide is bonded on said substrate.

2. The array as defined in claim 1, wherein said first single-stranded oligonucleotide has a functional group or bonding group at the 5′ terminal or 3′ terminal thereof, said first single-stranded oligonucleotide being bonded to said substrate through said functional group or bonding group.

3. The array as defined in claim 1, wherein said metal substrate is a transparent substrate having a surface layer formed of a thin gold layer.

4. The array as defined in claim 1, wherein said metal substrate includes a dimer-type alkane densely packed thereon, wherein said first single-stranded oligonucleotide is bonded to said metal substrate through direct or indirect bond with said dimer-type alkane.

5. The array as defined in claim 1, which is bonded to said substrate by use of a heterobifunctional hydrophilic polymer molecule expressed by a general formula of X—R—Y, wherein: X is a functional group on a surface of a solid surface or a functional group to be bonded to a functional group introduced to the surface of said solid surface; Y being a functional group to be bonded to a biomolecule (A); and R being a repeating unit of said polymer molecule.

6. The array as defined in claim 1, which has a background region on which a hydrophilic polymer molecule is immobilized.

7. The array as defined in claim 6, wherein said hydrophilic polymer molecule includes a plurality of metal-binding functional groups.

8. The array as defined in claim 6, wherein said hydrophilic polymer molecule has a plurality of branches, wherein said metal-binding functional groups are located at the terminals of at least a part of said branches.

9. The array as defined in claim 6, wherein said hydrophilic polymer molecule is ethylene glycol.

10. The array as defined in claim 1, which includes a marker indicative of a position of a spot.

11. The array as defined in claim 7, wherein said marker has a distinguishable character or numeral shape.

12. The array as defined in claim 7, wherein said marker is patterned in a monomolecular layer.

13. A method of preparing a double-stranded oligonucleotide array comprising the steps of (1) hybridizing a first single-stranded oligonucleotide and a second single-stranded oligonucleotide to form a double-stranded oligonucleotide having said first and second single-stranded oligonucleotides entirely or partially bonded together in a complementary manner, and (2) bonding a terminal of said first single-stranded oligonucleotide to a metal substrate to immobilize said double-stranded oligonucleotide formed in said step (1) on said metal substrate.

14. The method as defined in claim 13, wherein said first single-stranded oligonucleotide has a functional group or bonding group at the 5′ terminal or 3′ terminal thereof, wherein said method includes the step of bonding said first single-stranded oligonucleotide to said substrate through said functional group or bonding group.

15. The array as defined in claim 13, wherein said metal substrate is a transparent substrate, wherein said method includes the step of forming a thin gold layer on a surface of said metal substrate.

16. The array as defined in claim 13, which includes the steps of densely packing a bifunctional-type alkane on said metal substrate, and bonding said first single-stranded oligonucleotide to said metal substrate through direct or indirect bond with said bifunctional-type alkane.

17. A biomolecule interaction measuring method comprising the step of providing a double-stranded oligonucleotide array having a plurality of double-stranded oligonucleotides immobilized on a metal substrate, and measuring the interaction between said double-stranded oligonucleotides and a biomolecule or aggregate thereof, wherein each of said double-stranded oligonucleotides include a first single-stranded oligonucleotide and a second single-stranded oligonucleotide, said first and second single-stranded oligonucleotides being entirely or partially bonded together in a complementary manner to form said double-stranded oligonucleotide, wherein among said first and second single-stranded oligonucleotides, only said first single-stranded oligonucleotide is bonded to said substrate.

18. The method as defined in claim 17, wherein said first single-stranded oligonucleotide is bonded to said substrate by use of a cross-linking agent including a heterobifunctional hydrophilic polymer molecule expressed by a general formula of X—R—Y, wherein: X is a functional group on a surface of a solid surface or a functional group to be bonded with a functional group introduced to the surface of said solid surface; Y is a functional group to be bonded to a biomolecule (A); and R is a repeating unit of said polymer molecule.

19. The method as defined in claim 17, wherein said measurement is performed using an array which has a background region on which a hydrophilic polymer molecule is immobilized.

20. The method as defined in claim 17, wherein said measurement is performed using an array which includes a maker indicative of a spot.

21. A biomolecule interaction measuring method comprising measuring the interaction between a first biomolecule and a second biomolecule or aggregate thereof by use of a substrate with a solid surface having said first biomolecule immobilized thereon, wherein said first biomolecule is immobilized on said substrate using a cross-linking agent including a heterobifunctional hydrophilic polymer molecule expressed by a general formula X—R—Y, wherein: X is a functional group on a surface of a solid surface or a functional group to be bonded with a functional group introduced to the surface of said solid surface; Y is a functional group to be bonded with a biomolecule (A); and R is a repeating unit of said polymer molecule.

22. The method as defined in claim 21, wherein said heterobifunctional hydrophilic polymer molecule has a molecular weight of 200 to 20000.

23. The method as defined in claim 21, wherein said R of said heterobifunctional hydrophilic polymer molecule has a structure expressed by a repeating unit: —(—O—R₁—)ₙ—, wherein R₁ is an alkylene group, and n is an integer number in the range of 4 to 450.

24. The method as defined in claim 21, wherein said functional groups X and Y of said heterobifunctional hydrophilic polymer molecule are any two selected from the group consisting of an amino group, a carboxyl group, a succinimide group, a sulfonated succinimide group, a maleimide group, a thiol group, an aldehyde group, a vinyl group, an isocyanate group, an epoxy group, a hydrazine group and an azido group.

25. The method as defined in claim 21, wherein said solid surface comprises a thin gold layer formed on said substrate, said thin gold layer including a functional group which is introduced using a compound expressed by a general formula X′—R′—Y′, wherein X′ is a functional group reactive to said thin gold layer, Y′ is a functional group to be bonded with a heterobifunctional hydrophilic polymer molecule, and R′ is an organic group.

26. The method as defined in claim 21, wherein said substrate includes plural kinds of said first biomolecules immobilized thereon in an array arrangement.

27. The method as defined in claim 21, wherein said first biomolecule is nucleic acid.

28. The method as defined in claim 21, wherein the interaction between said first biomolecule and said second biomolecule or aggregate thereof is measured by utilizing surface plasmon resonance.

29. The method as defined in claim 21, wherein the interaction between said first biomolecule and said second biomolecule or aggregate thereof is measured through surface plasmon resonance imaging.

30. The method as defined in claim 21, wherein said second biomolecule is a protein.

31. The method as defined in claim 30, wherein said protein is a transfer factor.

32. The method as defined in claim 21, wherein said measurement is performed using an array which has a background region on which a hydrophilic polymer molecule is immobilized.

33. The method as defined in claim 21, wherein said measurement is performed using an array which includes a maker indicative of a spot.

34. An array for immobilizing a biomolecule or an aggregate of biomolecules on the surface thereof, comprising a immobilization area for immobilizing said biomolecule or said aggregate of biomolecules, and a background area other than said immobilization area, wherein said immobilization area includes a substance serving as an initial point of said immobilization, and said background area includes a hydrophilic polymer molecule immobilized thereon.

35. The array as defined in claim 34, wherein said background area includes a hydrophilic polymer molecule immobilized thereon.

36. The array as defined in claim 34, wherein said hydrophilic polymer molecule includes a plurality of metal-binding functional groups.

37. The array as defined in claim 34, wherein said hydrophilic polymer molecule has a plurality of branches, wherein said metal-binding functional groups are located at the terminals of at least a part of said branches.

38. The array as defined in claim 34, wherein said hydrophilic polymer molecule is ethylene glycol.

39. An array comprising a plurality of spots formed on a metal substrate in an array arrangement, and a plurality of markers indicative of the respective positions of said spots.

40. The array as defined in claim 35, wherein said marker has a distinguishable character or numeral shape.

41. The array as defined in claim 35, wherein said marker is patterned in a monomolecular layer.

42. An array comprising a plurality of first functional groups residing thereon, wherein a biomolecule having a second functional group is immobilized on said array through the reaction between a part of said first functional groups and said second functional groups, wherein the remaining first functional groups are blocked to preclude covalent bonding.


SEQUENCE LISTING (11 sequences)

SEQ ID NO: 1 - DNA, 31 bp, Artificial
  GATA reg anchor side
tttttttttttttttcggaatgataattacg

SEQ ID NO: 2 - DNA, 16 bp, artificial
  GATA reg complementary side
cgtaattatcattccg

SEQ ID NO: 3 - DNA, 31 bp, artificial
  GATA mut anchor side
tttttttttttttttcggaatagtaattacg

SEQ ID NO: 4 - DNA, 16 bp, artificial
  GATA mut complementary side
cgtaattatcattccg

SEQ ID NO: 5 - DNA, 25 bp, artificial
  anchor-side DNA, (n)13 is any recognition sequence
cggaatnnnnnnnnnnnnnttactc

SEQ ID NO: 6 - DNA, 13 bp, artificial
  MARE 25 recognition sequence
tgctgactcagca

SEQ ID NO: 7 - DNA, 14 bp, artificial
  hOPSIN recognition sequence
tgctgaattcagcc

SEQ ID NO: 8 - DNA, 13 bp, artificial
  HNQO1m recognition sequence
agttgactcagca

SEQ ID NO: 9 - DNA, 13 bp, artificial
  MARE 23 recognition sequence
caatgactcattg

SEQ ID NO: 10 - DNA, 13 bp, artificial
  hBglHS4 recognition sequence
ggctgactcactc

SEQ ID NO: 11 - DNA, 13 bp, artificial
  mGSTY recognition sequence
tggtgacaaagca